(19) United States
(12) Patent Application Publication
(10) Pub. No.: US 2025/0136907 A1
(43) Pub. Date: May 1, 2025

(54) SYSTEM, APPARATUS, AND METHOD FOR PROVIDING EX VIVO APPROXIMATION OF MICRO- AND/OR NANOPARTICLE ORAL TOXICITY IN HUMANS

(71) Applicant: Trustees of Tufts College, Medford, MA (US)

(72) Inventors: David L. Kaplan, Concord, MA (US); Ying Chen, Winchester, MA (US)

(21) Appl. No.: 18/824,487

(22) Filed: Sep. 4, 2024

Related U.S. Application Data
(63) Continuation of application No. PCT/US23/15068, filed on Mar. 13, 2023.
(60) Provisional application No. 63/269,235, filed on Mar. 11, 2022.
(60) Provisional application No. 63/323,007, filed on Mar. 23, 2022.

(51) Int. Cl. C12M 3/00; C12M 1/00; C12M 1/34; C12M 1/36; C12N 5/00; C12N 5/071; G16H 10/60; G16H 15/00; G16H 20/10

(52) U.S. Cl. CPC C12M 21/08; C12M 29/00; C12M 41/32; C12M 41/36; C12M 41/48; C12N 5/0068; C12N 5/0697; G16H 10/60; G16H 15/00; G16H 20/10; C12N 2533/50

(57) ABSTRACT

Systems and methods for providing ex vivo approximation of micro- and/or nanoparticle oral toxicity in humans include an ex vivo human intestinal model organoid comprising an epithelial layer that separates an apical volume and a basal volume of the ex vivo human intestinal model organoid, a bioreactor for maintaining the ex vivo human intestinal model organoid under physiologically relevant culture conditions, a particle administration inlet adapted to receive a predefined quantity of micro- and/or nanoparticles and to introduce the predefined quantity to the apical volume of the ex vivo human intestinal model organoid according to a predefined dosing regimen, an analytical platform adapted to: interrogate the ex vivo human intestinal model organoid or associated culture media within the bioreactor, and/or to receive and interrogate the ex vivo human intestinal model organoid and/or the associated culture media after removal from the bioreactor, a processor, and a memory.

### CLAIM TO PRIORITY

This application is a continuation of International Application Serial Number PCT/US2023/015068 (Attorney Docket No. 2095.0035), entitled “SYSTEM, APPARATUS, AND METHOD FOR PROVIDING EX VIVO APPROXIMATION OF MICRO-AND/OR NANOPARTICLE ORAL TOXICITY IN HUMANS,” filed Mar. 13, 2023, and published as WO2023172769.

International Application Serial Number PCT/US2023/015068 claims the benefit of priority to the following provisional applications, which are hereby incorporated by reference in their entirety: U.S. Ser. No. 63/269,235 filed Mar. 11, 2022 and entitled IMPACT OF MICROPLASTICS AND NANOPLASTICS ON A 3D TISSUE-ENGINEERED HUMAN INTESTINAL MODEL (Attorney Docket No. 2095.0033), and U.S. Ser. No. 63/323,007 filed Mar. 23, 2022 and entitled COMPARING POLYSTYRENE (PS) PARTICLE UPTAKE AND POTENTIAL TOXICITY IN REGULARLY DIFFERENTIATED HUMAN-DERIVED, IN VITRO ENTEROID (HIE) SYSTEMS TO M-CELL INDUCED HIE SYSTEMS (Attorney Docket No. 2095.0034).

### STATEMENT REGARDING FEDERALLY FUNDED RESEARCH

This invention was made with government support under grants NIH U19-AI131126 and NIH P41EB027062, both awarded by the National Institutes of Health. The government has certain rights in the invention.

### BACKGROUND

### Field

Disclosed herein are systems and methods for providing ex vivo approximation of micro- and/or nanoparticle oral toxicity in humans.

### Description of the Related Art

Micro- and nano-plastics (MPs and NPs) released from plastics in the environment can enter the food chain and target the human intestine. However, knowledge about the effects of these particles on the human intestine is still limited due to the lack of relevant human intestinal models to validate data obtained from animal studies or tissue models employing cancer cells. Much of the research concerning in vitro cellular uptake and cytotoxic impact of MPs and NPs in human intestinal epithelium has been carried out using monocultures of human cancerous cell lines, which have various limitations (e.g., much higher TEER values (1600-2500Ω·cm²) than what is reported for the intestine in vivo (50-100Ω·cm²) reflecting poorer permeability of compounds through the paracellular route, do not faithfully mirror the broad diversity of in vivo epithelial cell diversity and therefore fail to reproduce the complexity of in vivo cellular responses, cancerous cells have low or absent expression of some clinically relevant transporter proteins, etc.).

There remains a need for robust human-based in vitro experimental models that can predict in vivo outcomes of MP and NP pollution hazards in the human intestine and also provide a basis for subsequent particle management and control if needed.

### SUMMARY

Organoid technology is used to create near physiologically relevant cell culture models for the evaluation of the potential toxic effects of MPs and NPs varying in size (covering micro to nano scales), concentrations, and exposure periods on patient organoid-derived intestinal epithelia with different cellular complexities (with and without M cells), examining cellular uptake and translocation, and impact on barrier integrity and immune response (cytokine release).

An example system for providing ex vivo approximation of micro- and/or nanoparticle oral toxicity in humans includes an ex vivo human intestinal model organoid comprising an epithelial layer that separates an apical volume and a basal volume of the ex vivo human intestinal model organoid, a bioreactor for maintaining the ex vivo human intestinal model organoid under physiologically relevant culture conditions, a particle administration inlet adapted to receive a predefined quantity of micro- and/or nanoparticles and to introduce the predefined quantity to the apical volume of the ex vivo human intestinal model organoid according to a predefined dosing regimen, and an analytical platform adapted to: interrogate the ex vivo human intestinal model organoid or associated culture media within the bioreactor; and/or to receive and interrogate the ex vivo human intestinal model organoid and/or the associated culture media after removal from the bioreactor, and a processor and a memory. The memory has stored thereon instructions that, when executed by the processor, cause the processor to: a) optionally send a signal to the particle administration inlet, thereby automatically controlling the predefined dosing regimen, b) at one or more predetermined times relative to the predefined dosing regimen, using the analytical platform, imaging the ex vivo human intestinal model organoid to identify localization of the predefined quantity of micro- and/or nanoparticles, measuring tight-junction degradation within the ex vivo human intestinal model organoid optionally by measuring ZO-1 expression levels, measuring epithelial integrity of the ex vivo human intestinal model organoid, and/or measuring cytokine upregulation within the ex vivo human intestinal model organoid, and c) generating a report including acquired data relating to the localization of the predefined quantity of micro- and/or nanoparticles within the ex vivo human intestinal model organoid, the tight-junction degradation within the ex vivo human intestinal model organoid, the epithelial integrity of the ex vivo human intestinal model organoid, and/or the cytokine upregulation within the ex vivo human intestinal model organoid, wherein the ex vivo human intestinal model organoid comprises either: an M cell concentration of between 0.1% and 10% within the epithelial layer; or an initial transepithelial electrical resistance of between 0 Ω·cm² and 1500 Ω·cm².

An example method of ex vivo approximation of micro- and/or nanoparticle oral toxicity using an ex vivo human intestinal model organoid comprising an epithelial layer that separates an apical volume and a basal volume of the ex vivo human intestinal model organoid includes a) introducing a predefined quantity of micro- and/or nanoparticles into the apical volume of the ex vivo human intestinal model organoid in a predefined dosing regimen under physiologically relevant culture conditions, b) at one or more predetermined times relative to the predefined dosing regimen, imaging the ex vivo human intestinal model organoid to identify localization of the predefined quantity of micro- and/or nanoparticles, measuring tight-junction degradation within the ex vivo human intestinal model organoid optionally by measuring ZO-1 expression levels, measuring epithelial integrity of the ex vivo human intestinal model organoid, and/or measuring cytokine upregulation within the ex vivo human intestinal model organoid, and c) generating a report including acquired data relating to the localization of the predefined quantity of micro- and/or nanoparticles within the ex vivo human intestinal model organoid, the tight-junction degradation within the ex vivo human intestinal model organoid, the epithelial integrity of the ex vivo human intestinal model organoid, and/or the cytokine upregulation within the ex vivo human intestinal model organoid, wherein the ex vivo human intestinal model organoid comprises either: an M cell concentration of between 0.1% and 10% within the epithelial layer; or an initial transepithelial electrical resistance of between 0Ω·cm² to 1500 Ω·cm².

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. The disclosure and the following detailed description of certain embodiments thereof may be understood by reference to the following figures:

FIG. 1 depicts a system for providing ex vivo approximation of micro- and/or nanoparticle oral toxicity in humans.

FIG. 2 depicts a system for providing ex vivo approximation of micro- and/or nanoparticle oral toxicity in humans.

FIG. 3A and FIG. 3B depict methods for providing ex vivo approximation of micro- and/or nanoparticle oral toxicity in humans.

FIG. 4 Establishment and characterization of SDM- and MDM differentiated monolayer cell cultures from intestinal organoids. (A-C) Organoid culture and the monolayer formation. Organoid (A1, B) were expanded, enzymatically digested into singlets and doublets (A2) and seeded on transwell inserts to form confluent monolayer (A3, C) for differentiation without M cells (A4) and with M cells (A5).

FIG. 5 TEER measurements on SDM-treated (A) and MDM-treated (B) monolayer cultures derived from patient intestinal organoids. (*p<0.05, ** p<0.005, *** p<0.001).

FIG. 6 Schematic of the experimental timeline of cell seeding, monolayer differentiation and particle dosing.

FIG. 7 (A-D) TEER measurements of SDM-treated organoid derived epithelial barriers with exposure to 1 μm MPs (A), 500 nm MPs (B), 100 nm NPs (C), and 30 nm NPs (D) at concentrations of 1,000 μg/mL, 500 μg/mL and 250 μL for up to 4 days (96 hours) post exposure. (E-P) Cytokine profiling of the SDM-treated cultures following exposure to 250 μg/mL, 500 μg/mL and 1000 μg/mL were conducted 24, 48, 72 and 96 hours. Inflammatory cytokines including TNF-α (E, I, M), TGF-β1 (F, J, N), IL-6 (G, K, O), and IL-8 (H, L, P) were measured and analyzed. The level of cytokines in the cell supernatant were determined using ELISA. (*p<0.05, ** p<0.005, *** p<0.001).

FIG. 8 (A-D) TEER measurements of MDM-treated organoid derived epithelial barriers with exposure to 1 μm MPs (A), 500 nm MPs (B), 100 nm NPs (C), and 30 nm NPs (D) at concentrations of 1,000 μg/mL, 500 μg/mL and 250 μL for up to 4 days (96 hours) post exposure. (E-P) Cytokine profiling of the MDM-treated cultures following exposure to 250 μg/mL, 500 μg/mL and 1000 μg/mL were conducted 24, 48, 72 and 96 hours. Inflammatory cytokines including TNF-α (E, I, M), TGF-β1 (F, J, N), IL-6 (G, K, O), and IL-8 (H, L, P) were measured and analyzed. The level of cytokines in the cell supernatant were determined using ELISA. (*p<0.05, ** p<0.005, *** p<0.001).

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices, and methods relating to surface patterning are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

Disclosed herein are systems and methods for providing ex vivo approximation of micro- and/or nanoparticle oral toxicity in humans. The systems and methods disclosed herein are useful in determining or predicting the biological effects of polystyrene micro- and nano-plastics on human intestinal organoid-derived epithelial tissue models without and with M cells. Human intestinal organoids can be used to develop epithelia to mimic the cell complexity and functions of native tissue (e.g., appropriate expression of a panel of metabolically essential genes and some critical genes involved in fatty acid and cholesterol homeostasis in the small intestine; more human-relevant prediction (than Caco-2 cells, for example) of the adverse effects caused by exposures to foreign compounds) and to study intestinal nutrient transport along with uptake and metabolism of drugs and exogenous compounds. Microfold cells (M cells) may be induced to distinguish their role when exposed to MPs and NPs. During the exposure, the M cells may act as sensors, capturers, and transporters of larger-sized particles and the epithelial cells may internalize the particles in a size-, concentration-, and time-dependent manner. In an example, high concentrations of particles may trigger the secretion of a panel of inflammatory cytokines linked to human inflammatory bowel disease (IBD).

In the systems and methods, if a barrier monolayer, such as an endothelial or epithelial monolayer, is disrupted, measures of transepithelial/transendothelial electrical resistance (TEER) may fall to very low levels (e.g., less than 10% of the healthy TEER value) and may be indicative of toxicity. Further, a significant percentage (e.g., >5%) of cells dying may indicate toxicity. In some cases, these results may be calibrated to generate particle-related dose-response curves related to TEER and/or cell viability (e.g., changes in live and dead cell number and the number of M cells in the epithelium, changes in cytokine profiles, such as the upregulations of proinflammatory cytokines) as a qualitative estimate of toxicity.

The systems and methods disclosed herein relate in part to a platform for predicting the potential risks of MP and NP exposure comprising human intestinal organoids, which may further be useful to: 1) establish and characterize organoid-derived intestinal epithelial monolayer models: monolayers without M cells (enterocytes, goblet cells, EECs, and Paneth cells) and monolayers with M cells (enterocytes, goblet cells, EECs, Paneth cells, and M cells); 2) compare the dynamics of cellular uptake and translocation of different sizes of MPs and NPs over time in these two different tissue models; 3) evaluate the potential dose-, size- and exposure period-dependent cytotoxic effects of the plastic particles on the intestinal barrier integrity and immune response in the tissue models; and 4) distinguish the role of M cells regarding the transport of plastic particles across the intestinal epithelium and how M cells shape specific immune responses to these particles. Throughout this disclosure, polystyrene (PS) MPs and NPs are described in the example methods and systems given their wide availability in a variety of sizes (e.g., 1 μm, 500 nm, 100 nm, and 30 nm), stability in cell culture media, and ease of localization and tracking in cell compartments via fluorescent labelling, but it should be understood that any type of MP or NP may be used in the disclosure, such as polypropylenes, polyethylenes, and the like.

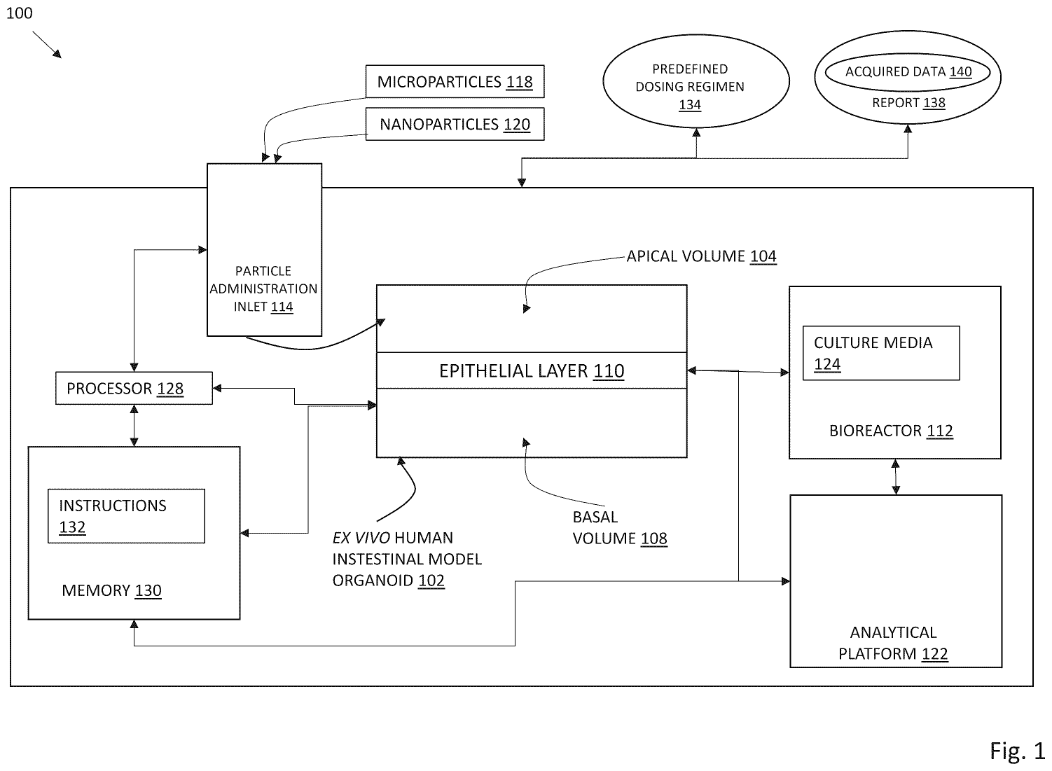

Referring now to FIG. 1, a system 100 for providing ex vivo approximation of micro- and/or nanoparticle oral toxicity in humans may include an ex vivo human intestinal model organoid 102 comprising an epithelial layer 110 that separates an apical volume 104 and a basal volume 108 of the ex vivo human intestinal model organoid 102. The system 100 may also include a bioreactor 112 for maintaining the ex vivo human intestinal model organoid 102 under physiologically relevant culture conditions. The system 100 may further include a particle administration inlet 114, such as an automated particle hopper or a manual hopper, adapted to receive a predefined quantity of microparticles 118 and/or nanoparticles 120 and to introduce the predefined quantity to the apical volume 104 of the ex vivo human intestinal model organoid 102 according to a predefined dosing regimen 134.

In embodiments, the particle administration inlet 114 may be an automated motorized hopper to dispense particles may provide a precise and controllable way to dispense particles. A hopper container of the automated motorized hopper may be a vessel that holds the particles to be dispensed and may comprise a material that is durable enough to withstand the mechanical stresses of the dispensing process. The size of the container may be selected based on the volume of particles that need to be dispensed. The motor of the automated motorized hopper provides the mechanical force to move the particles out of the hopper container and into the dispensing mechanism. A DC motor or stepper motor can be used for this purpose, depending on the required torque and speed. The auger of the automated motorized hopper may be a helical screw that is powered by the motor and rotates inside the hopper container to move the particles toward the dispensing mechanism. The size and pitch of the auger may be selected based on the size and flow properties of the particles being dispensed. A dispensing mechanism, such as a slide gate, rotary valve, or vibratory feeder, of the automated motorized hopper may release the particles from the hopper container. The dispensing mechanism may provide a consistent flow of particles and be adjustable to control the dispensing rate. A control system of the automated motorized hopper may regulate the operation of the motor and dispensing mechanism. It can be a simple on/off switch or a more sophisticated system that allows for precise control over the dispensing rate and volume.

In embodiments, the particle administration inlet 114 may be a manual hopper to dispense particles and may include the hopper container, dispensing mechanism, flow control, and handle. The dispensing mechanism may comprise a simple hand-operated gate or a metering valve that can be adjusted to control the dispensing rate. The flow control mechanism may help regulate the rate of particle flow. For example, the hopper can be equipped with a vibration mechanism that helps to evenly distribute the particles in the hopper and promote a consistent flow rate. The hopper can be designed with a handle to make it easy to carry and maneuver. The handle can also be used to control the flow of particles by adjusting the position of the dispensing mechanism.

Continuing with reference to FIG. 1, the system 100 may also include an analytical platform 122 adapted to: interrogate the ex vivo human intestinal model organoid 102 or associated culture media 124 within the bioreactor 112, and/or to receive and interrogate the ex vivo human intestinal model organoid 102 and/or the associated culture media 124 after removal from the bioreactor 112. The analytical platform may include at least one of a transepithelial electrical resistometer, a confocal laser scanning microscope, a multiphoton microscope, a wide-field fluorescence microscope, a super-resolution microscope, qRT-PCR, a scanning electron microscope, live and dead staining capabilities and assays, an assay, an ELISA assay for cytokine upregulation, an ELISA assay for tumor necrosis factor (TNF)-α, tumor growth factor (TGF)-β1, interleukin (IL)-6, and/or IL-8, or a scanning electron microscope.

In embodiments, TEER (Trans-Epithelial Electrical Resistance) meters use electrodes to apply an electrical current across electrodes placed on both sides of a cellular monolayer and measure voltage and current to calculate the electrical resistance of the barrier. Traditionally, TEER is measured by placing two elongated electrodes on either side of a transwell insert with a confluent cell layer. After the TEER measurement is performed using a TEER meter, the measured TEER values are typically displayed on the meter's display screen. Another way to measure TEER includes using labelled compound transport across the barrier, such as labelled dextrans of different molecular weights with a fluorophore. The concentration of the label on both sides of the membrane/epithelial barrier is measured over time to determine barrier properties.

The system 100 may include a processor 128 and a memory 130. The memory 130 has instructions 132 stored thereon that, when executed by the processor 128, cause the processor 128 to optionally send a signal to the particle administration inlet 114, thereby automatically controlling the predefined dosing regimen 134. The instructions 132 may also cause the processor 128 to, at one or more predetermined times relative to the predefined dosing regimen 134, using the analytical platform 122, image the ex vivo human intestinal model organoid 102 to identify localization of the predefined quantity of microparticles 118 and/or nanoparticles 120, measure tight-junction degradation within the ex vivo human intestinal model organoid 102 optionally by measuring ZO-1 expression levels, measure epithelial integrity of the ex vivo human intestinal model organoid 102, and/or measure cytokine upregulation within the ex vivo human intestinal model organoid 102. The instructions 132 may also cause the processor 128 to generate a report 138 including acquired data 140 relating to the localization of the predefined quantity of microparticles 118 and/or nanoparticles 120 within the ex vivo human intestinal model organoid 102, the tight-junction degradation within the ex vivo human intestinal model organoid 102, the epithelial integrity of the ex vivo human intestinal model organoid 102, and/or the cytokine upregulation within the ex vivo human intestinal model organoid 102. In embodiments, the ex vivo human intestinal model organoid 102 may include either: an M cell concentration of between 0.1% to 10% of the total epithelial cell population within the epithelial layer 110; or an initial transepithelial electrical resistance of between 0 Ω·cm² to 1500 Ω·cm².

In some embodiments, the M cell concentration is at least 0.1%. In some embodiments, the M cell concentration is less than 10%. In some embodiments, the M cell concentration is between 0.1% and 10%. In some embodiments, the M cell concentration is at least 2%. In some embodiments, the M cell concentration is at least 4%. In some embodiments, the M cell concentration is less than 8%. In some embodiments, the M cell concentration is less than 6%. In some embodiments, the M cell concentration is between 2% and 8%. In some embodiments, the M cell concentration is between 4% and 6%.

In embodiments, the initial transepithelial electrical resistance is between 022·cm² to 1500 Ω·cm². In some embodiments, the initial transepithelial electrical resistance is less than 1500 Ω·cm². In some embodiments, the initial transepithelial electrical resistance is between 50 Ω·cm² to 500 Ω·cm². In some embodiments, the initial transepithelial electrical resistance is at least 50 Ω·cm². In some embodiments, the initial transepithelial electrical resistance is less than 500 Ω·cm². In some embodiments, the initial transepithelial electrical resistance is between 200 Ω·cm² to 1200 Ω·cm². In some embodiments, the initial transepithelial electrical resistance is at least 200 Ω·cm². In some embodiments, the initial transepithelial electrical resistance is less than 50 Ω·cm². In some embodiments, the initial transepithelial electrical resistance is between 400 Ω·cm² to 600 Ω·cm². In some embodiments, the initial transepithelial electrical resistance is at least 400 Ω·cm². In some embodiments, the initial transepithelial electrical resistance is less than 600 Ω·cm².

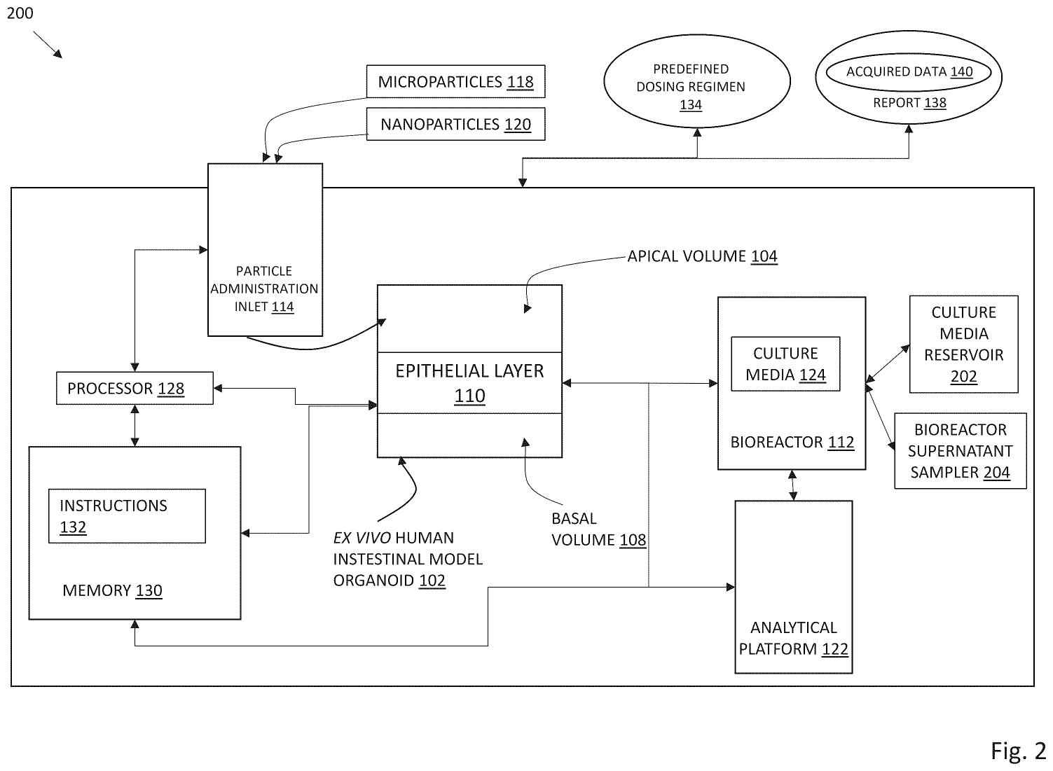

In embodiments, and referring to FIG. 2, in a system 200, the bioreactor 112 may be coupled to a culture media reservoir 202 and the system 200 may be adapted to regenerate and/or circulate the culture media 124 within the bioreactor 112 to maintain the physiologically relevant culture conditions. The system 200 may further include a bioreactor supernatant sampler 204 operable to extract a culture media supernatant sample from the bioreactor 112. The bioreactor supernatant sampler 204 may further be operable to deliver the culture media supernatant sample to the analytical platform 122 and/or the assay and/or the ELISA assay.

In embodiments, the instructions 132, when executed by the processor 128, further cause the processor 128 to associate at least one of the localization, the tight-junction degradation, the epithelial integrity, or the cytokine upregulation with a predicted in vivo toxicity for the microparticles 118 or nanoparticles 120.

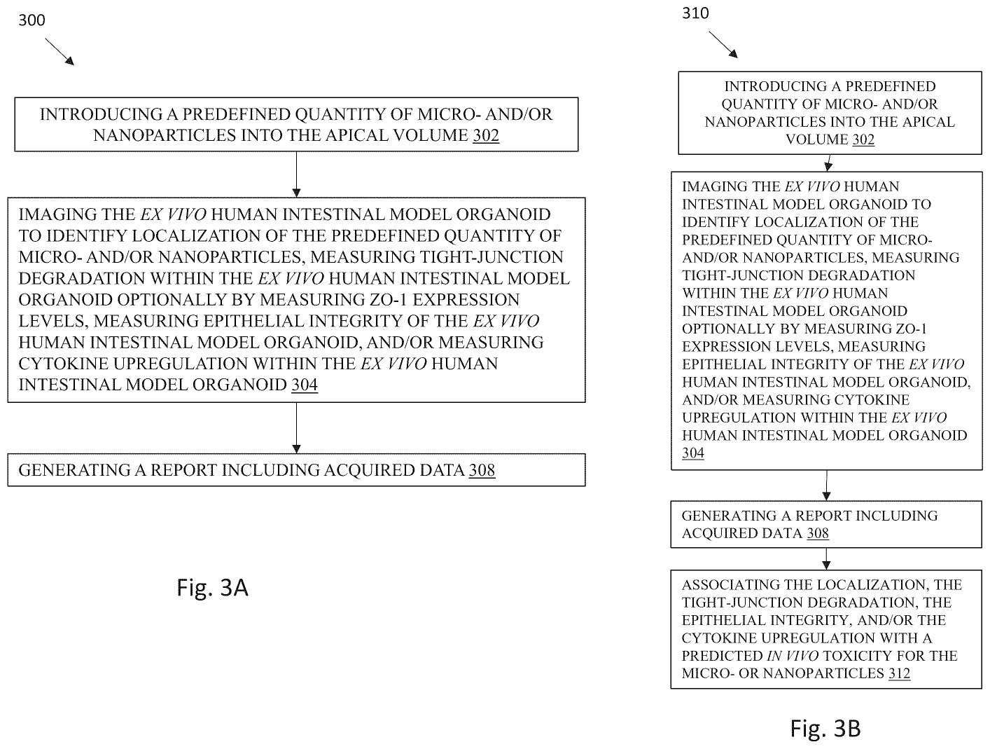

Referring now to FIG. 3A, a method 300 of ex vivo approximation of micro- and/or nanoparticle oral toxicity using an ex vivo human intestinal model organoid comprising an epithelial layer that separates an apical volume and a basal volume of the ex vivo human intestinal model organoid may include a step 302 of introducing a predefined quantity of micro- and/or nanoparticles into the apical volume of the ex vivo human intestinal model organoid in a predefined dosing regimen under physiologically relevant culture conditions. The method 300 may further include a step 304 of at one or more predetermined times relative to the predefined dosing regimen, at least one of imaging the ex vivo human intestinal model organoid to identify localization of the predefined quantity of micro- and/or nanoparticles, measuring tight-junction degradation within the ex vivo human intestinal model organoid optionally by measuring ZO-1 expression levels, measuring epithelial integrity of the ex vivo human intestinal model organoid, or measuring cytokine upregulation within the ex vivo human intestinal model organoid. The method 300 may further include a step 308 of generating a report including acquired data relating to the localization of the predefined quantity of micro- and/or nanoparticles within the ex vivo human intestinal model organoid, the tight-junction degradation within the ex vivo human intestinal model organoid, the epithelial integrity of the ex vivo human intestinal model organoid, and/or the cytokine upregulation within the ex vivo human intestinal model organoid, wherein the ex vivo human intestinal model organoid comprises either: an M cell concentration of between 0.1% to 10% within the epithelial layer; or an initial transepithelial electrical resistance of between 0 Ω·cm² to 1500 Ω·cm². In some embodiments, and referring to FIG. 3B, a method 310 may further include a step 312 of associating the localization, the tight-junction degradation, the epithelial integrity, and/or the cytokine upregulation with a predicted in vivo toxicity for the micro- or nanoparticles. The ex vivo human intestinal model organoid may include the M cell concentration of between 0.1% to 10% within the epithelial layer. The ex vivo human intestinal model organoid may include the M cell concentration of between 2% to 8% within the epithelial layer. In embodiments, the ex vivo human intestinal model organoid may include the M cell concentration of between 4% to 6% within the epithelial layer. In some embodiments, the M cell concentration is at least 0.1%. In some embodiments, the M cell concentration is less than 10%. In some embodiments, the M cell concentration is at least 2%. In some embodiments, the M cell concentration is at least 4%. In some embodiments, the M cell concentration is less than 8%. In some embodiments, the M cell concentration is less than 6%.

In embodiments, the ex vivo human intestinal model organoid may include the initial transepithelial electrical resistance of between 0 Ω·cm² to 1500 Ω·cm². In embodiments, the ex vivo human intestinal model organoid may include the initial transepithelial electrical resistance of between 200 Ω·cm² to 1200 Ω·cm². In embodiments, the ex vivo human intestinal model organoid may include the initial transepithelial electrical resistance of between 40002·cm² to 600 Ω·cm². In some embodiments, the initial transepithelial electrical resistance is less than 1500 Ω·cm². In some embodiments, the initial transepithelial electrical resistance is between 500·cm² to 500 Ω·cm². In some embodiments, the initial transepithelial electrical resistance is at least 50 Ω·cm². In some embodiments, the initial transepithelial electrical resistance is less than 500 Ω·cm². In some embodiments, the initial transepithelial electrical resistance is at least 200 Ω·cm². In some embodiments, the initial transepithelial electrical resistance is less than 550 Ω·cm². In some embodiments, the initial transepithelial electrical resistance is at least 400 Ω·cm². In some embodiments, the initial transepithelial electrical resistance is less than 600 Ω·cm².

In embodiments, the ex vivo human intestinal model organoid may be a two-dimensional human intestinal model organoid. In embodiments, the ex vivo human intestinal model organoid is a three-dimensional human intestinal model organoid. In embodiments, the ex vivo human intestinal model organoid is patient-specific and the report includes patient-specific data or patient-specific predictions. As used herein, patient-specific refers to a model that is at least partly based on cells that have been harvested from a subject and optionally reprogrammed according to methods known in the art.

In embodiments, imaging may be performed using a scanning electron microscope, a confocal laser scanning microscope, or a combination thereof. In embodiments, measuring the epithelial integrity includes measuring transepithelial electrical resistance. In embodiments, measuring cytokine upregulation may be by performing an assay, such as an ELISA assay. In embodiments, measuring cytokine upregulation may be by measuring TNF-α, TGF-β1, IL-6, and/or IL-8 upregulation.

In embodiments, the micro- and/or nanoparticles may be at least one of partly polymeric or partly metallic. Particles may comprise natural materials, synthetic materials, metals, polymers, composites, or the like. Particles may comprise different sizes, doses, shapes, chemistry, surface coatings, or the like. Particles may be labelled or unlabeled. Particles may be hollow, porous, or dense.

In embodiments, the ex vivo human intestinal model organoid may comprise cells grown and/or differentiated on a silk fibroin-based tissue scaffold.

### Example

### Materials and Methods

PS MPs and NPs (sources, size, fluorescence tags) and characterization.

Commercial fluorescent PS micro- and nano-plastic particles (PS MPs NPs) in varying sizes were purchased from Millipore Sigma (Burlington, MA): 1 μm, 500 nm, 100 nm labeled with red fluorescence and 30 nm labeled with yellow-green fluorescence. To characterize and evaluate PS particles, 25 μg/mL particle solutions were prepared at 1:1000 dilution in DPBS (Gibco, Waltham, MA).

To confirm sizes, 50 μL of PS MPs and NPs solution were placed on SEM conductive tape, dried, coated with ˜10 nm of platinum using a sputter coater (208 HR, Cressington Scientific Instruments Inc., Cranberry Twp) in accordance with previous procedures, and imaged at ˜2-3 kV on a Zeiss Ultra Plus Scanning Electron Microscope (Carl Zeiss, Oberkochen, Germany).

Cell models.

Intestinal organoid culture-Human intestinal organoids, isolated from tissue biopsies of the human ileum (Baylor College of Medicine-Texas Medical Center Digestive Diseases Center Enteroid Core) were cultured according to previously established methods.[9] These intestinal organoids have been well characterized and used in basic and disease research.[10-13] Briefly, cryovials containing organoids were thawed under cool tap water and resuspended in CMGF-(conditioned medium without growth factors) medium (Advanced DMEM/F12 (Invitrogen), 1× GlutaMAX (Invitrogen), and 10 mM HEPES buffer (Invitrogen, Waltham, MA)). Cells were centrifuged at 1200 rpm for 5 minutes at 4° C., resuspended in Matrigel (Corning, Corning, NY), and plated in droplets using the hanging drop method (2×15 μL/well) on cell-culture treated 24-well plates (ThermoFisher, Waltham, MA). After incubation, samples were fed (500 μL/well) with conditioned Wnt/R-spondin/Noggin (CWRN) growth medium (Advanced DMEM/F12 (Invitrogen) supplemented with 100 U/mL penicillin-streptomycin (Invitrogen), 10 mM HEPES buffer (Invitrogen), 1× GlutaMAX (Invitrogen), 50 ng/mL epidermal growth factor (EGF) (Invitrogen), 10 mM nicotinamide (Sigma-Aldrich), 10 nM gastrin I (Sigma-Aldrich), 500 nM A-83-01 (Tocris Bioscience, Bristol, United Kingdom), 10 μM SB202190 (Sigma-Aldrich, St. Louis, MO), 1×B27 supplement (Invitrogen), 1×N2 supplement (Invitrogen), and 1 mM N-acetylcysteine (Sigma-Aldrich)) and 10 μM of Rock Inhibitor Y-27632 (Sigma-Aldrich) and left to incubate at 37° C. (5% CO₂) for two days. Media were refreshed every other day with CWRN medium (without Y-27632) until ˜80% confluence was reached (˜3-5 days, checked via light microscopy). Organoids were subsequently passaged using an established protocol of mechanoenzymatic degradation with TrypLE Express (Thermo Fisher Scientific) in typical ratios of 1:3 or 1:4 before monolayer preparation to ensure appropriate confluence and “stemness” (checked via light microscopy). Passages 14-42 were used for experiments.

Cell seeding to form monolayers-Once confluent, organoids were collected and dissociated into single cells and seeded on 0.4 μm pore polycarbonate membrane inserts (Costar, Washington, D.C.) as previously described.[9] Briefly, to allow for proper cell adherence, apical chambers of each transwell were coated with 200 μL diluted collagen solution (First Link, United Kingdom) and incubated for at least two hours at 37° C. After incubation, collagen dilutions were aspirated and left to dry with lids slightly ajar before beginning the seeding protocol. Organoids were washed with DPBS thrice and treated with 500 μL of 0.5 μM EDTA (Lonza, Basel, Switzerland). Matrigel droplets were thoroughly scratched and disrupted physically with a P1000 pipette tip through pipetting 10 times to help break up the cell-Matrigel extracellular matrix. Samples were transferred to 15 mL conical tubes (6 wells/tube) and centrifuged at 1200 rpm at 4° C. for 5 minutes. Supernatants were aspirated and cell pellets were resuspended in 0.5 mL of 0.05% Trypsin-EDTA (Gibco, 25300-062), then placed in a water bath (37° C.) for ˜3.5-4 minutes to help induce enzymatic digestion. To halt trypsin enzymatic activity, double the amount (1 mL) of 10% FBS (Gibco) in CMGF—was added to each tube. Contents were triturated with a P1000 micropipette ˜40-50 times to further dissociate organoids into single cells. Proper dispersion was checked under a light microscope. Resulting cell solutions strained through a 40 μm cell strainer (Corning) and centrifuged at 1200 rpm for 5 minutes. Supernatants were aspirated and cell pellets were resuspended in conditioned CWRN medium and 10 μM Y-27632. 200 μL of resulting cell solutions were added to the apical chamber of each transwell. 600 μL of CWRN and 10 μM Y-27632 was added to the basolateral compartments of each transwell. After two days, spent media was aspirated out and cells were fed with CWRN medium every other day until confluence was reached (˜24-48 hours post-seeding).

Monolayer differentiation without M cells, with M cells-Once confluent in CWRN growth medium (˜24-48 hours), intestinal monolayers derived from the organoids were induced to differentiate utilizing standard differentiation medium (SDM, CMGF-supplemented with 5% Noggin, 50 ng/mL epidermal growth factor (EGF) (Invitrogen), 10 mm nicotinamide (Sigma-Aldrich), 10 nm gastrin I (Sigma-Aldrich), 1×B27 supplement (Invitrogen), 1×N2 supplement (Invitrogen), 1 mM N-acetylcysteine (Sigma-Aldrich), and 50 μg/mL Primocin (Invitrogen) or M cell differentiation medium (MDM, SDM supplemented 100 ng/ml recombinant Human RANKL (Peprotech, Cranbury, NJ) and with 50 ng/mL recombinant Human TNF-α (Gibco)).[14] For cell differentiation, CWRN medium was carefully aspirated from each chamber and replaced with 200 μL of SDM to the apical chamber, and 600 μL of SDM or 600 μL of MDM were added to the basal chamber according to the condition.

MPs and NPs administration, sample collection and characterization.

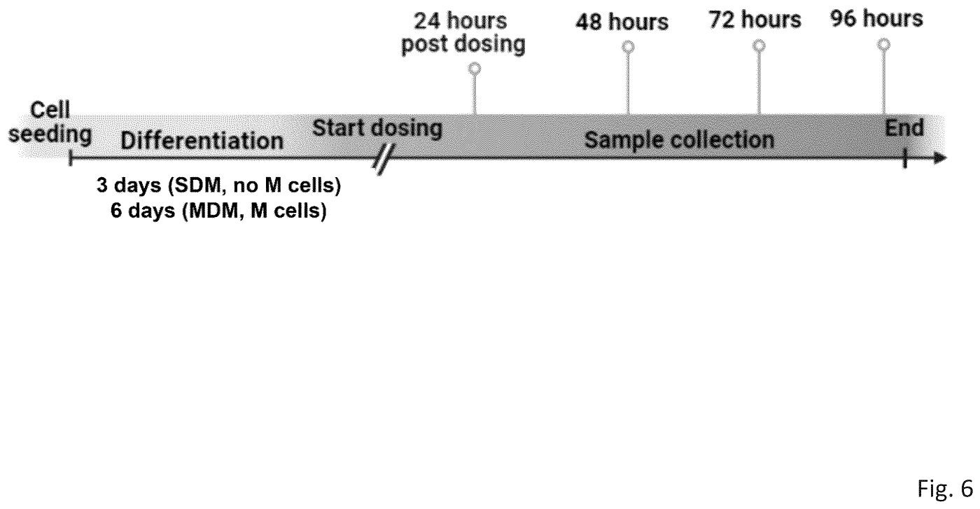

Assessment of cell barrier integrity by Transepithelial Electrical Resistance (TEER)—To evaluate the effect of different PS particle sizes (30 nm, 100 nm, 500 nm, and 1 μm), and concentrations (0 μg/mL, 250 μg/mL, 500 μg/mL, and 1000 μg/mL) on organoid-derived monolayer systems, TEER measurements were taken daily after differentiation media introduction and exposure (FIG. 6). Measurements were conducted as follows: the electrode for an Epithelial Voltohmeter (EVOM2, World Precision Instruments, Sarasota, FL) was briefly sterilized in 70% ethanol for 15 minutes, dried, and preconditioned in standard growth medium for 30 minutes. Once the meter reading hit 0, the EVOM2 was switched to read ohms, and TEER measurements were read across chambers of transwell samples.

Immunostaining of ZO-1, LYZ, SI, ChgA, Muc2, GP2-After exposure to PS MPs NPs at different sizes, samples (including the control group) were fixed in 4% PFA for 30 minutes and washed thrice with DPBS. Samples were then permeabilized using 0.01% Triton X-100 (Thermo Fisher, A16046AP) and 1% BSA (Sigma Aldrich A7906) in DPBS (Gibco 14190-144) for 30-45 minutes followed by blocking with 3% BSA in DPBS for one hour at room temperature. Transwell membranes were briefly washed with DPBS thrice after permeabilization and blocking steps. The primary antibodies: Anti-ZO-1 (Invitrogen), Anti-Lysozyme (Abcam, Cambridge, Massachusetts), Anti-human SI (sucrase-isomaltase, Santa Cruz Biotechnology, Dallas, TX), Anti-Chg-A (Chromogranin A, Abcam), Anti-human Muc-2 (Santa Cruz Biotechnology), and Anti-GP2 (MBL International, Woburn, Massachusetts) was diluted 1:100 in 0.1% BSA, added at a volume of 200 μL/apical chamber of transwells, and stored at 4° C. overnight. Following incubation, samples were washed (3×DPBS) and 200 μL of DAPI (diluted 1:1 in 0.1% BSA) and appropriate secondary antibody (diluted 1:200 in 0.1% BSA) was added. Secondary Antibodies used in the experiments are Alexa Fluor™ 488 Goat anti-Rabbit IgG (H+L) Cross-Adsorbed Secondary (Invitrogen), Alexa Fluor™ 488 Goat anti-Mouse IgG (H+L) Cross-Adsorbed Secondary Antibody (Invitrogen), Alexa Fluor™ 594 Donkey anti-Mouse IgG (H+L) Highly Cross-Adsorbed Secondary Antibody (Fisher Scientific), Alexa Fluor™ Plus 647 Goat anti-Rabbit IgG (H+L) Highly Cross-Adsorbed Secondary Antibody (Invitrogen), and Alexa Fluor™ 647 Goat anti-Mouse IgG (H+L) Highly Cross-Adsorbed Secondary Antibody (Invitrogen). Samples were incubated at room temperature for 30 minutes to 1 hour followed by rinsing (3×DPBS) and stored in DPBS until imaging. Imaging was conducted using a Leica SP8 confocal microscope (Leica Microsystems) within 48 hours using filter sets for DAPI (Ex/Em: 350/470 nm), GFP/FITC (Ex/Em: 488/514 nm), Texas Red (Ex/Em: 540/605 nm), and Cy5 (Ex/Em: 651/670). Organoid monolayers treated with fluorescent red 1 μm, 500 nm, and 100 nm PS-MNPs were stained with 488 nm and fluorescing antibodies, while samples treated with yellow-green 30 nm PS-MNPs were stained with 594 nm and fluorescing antibodies.

In general, intestinal epithelium with integral barrier was identified by staining of ZO-1 antibody, M cell was stained by GP2 antibody, and cell nuclei were stained with DAPI.

Scanning electron microscope (SEM)—Organoids monolayers with and without the treatment of the plastic particles in STD or MDM were prepared for SEM after TEER values stabilized and post exposure. Samples were crosslinked with 1% glutaraldehyde (GA, Sigma) in DPBS and incubated for one hour. GA was aspirated, samples were washed with DBS (3×, 2 minutes each) and incubated at 4° C. for at most a week. Subsequently, DPBS was aspirated from transwell samples and replaced with two washes with distilled ultrapure water (2 minutes each, Invitrogen). Samples were then prepared with a gradual dehydration process using dilutions of 200 proof ethanol (Fisher Bioreagents, Waltham, MA), 25% for 10 minutes on ice, 50% for 10 minutes on ice, 75% for 15 minutes at −20° C., 95% for 15 minutes at −20° C., and 100% for 15 minutes at ˜20° C. Transwell membranes were carefully excised and placed on covered glass slips. A critical point dryer (AutoSamdri-815) and sputter coater (208 HR, Cressington Scientific Instruments Inc., Cranberry Twp) were used to prepare samples for microscopy. The Zeiss Ultra Plus Scanning Electron Microscope (Carl Zeiss, Oberkochen, Germany) was set to ˜2-3 kV and images were obtained.

### Cytokine Profiling

To assess cytokine release in the samples, media samples were collected for testing. Human TNF-α, TGF-β1, IL-6 and IL-8 cytokine kits (Invitrogen) were used according to manufacturer instructions and previously reported.[15] Briefly, antibody-coated 96 microwell plates were washed with wash buffer (provided in kit). 100 μL of sample blanks and six standards (1.56 pg/mL, 3.13 pg/mL, 6.25 pg/mL, 12.50 pg/mL, 25 μg/mL, 50 μg/mL and 100 μg/mL) were prepared and arranged in the first two columns of the coated 96 microwell plate, and 100 μL of organoid-derived monolayer transwell triplicates treated without and with PS MPs and NPs arranged in remaining columns. Assay buffer was added to each microwell prepared for testing. The microwell plate was subject to two separate incubations (and subsequent washes) with a Biotin-conjugate and streptavidin-HRP, two molecules used to attach to the precoated antibodies. Finally, 3,3′,5,5′-Tetramethylbenzidine (TMB) substrate (a colorimetric solution), was added to each well for 10 minutes and enzymatic reactions were halted with the stop solution. Absorbance measurements were read from each well at 450 nm, and a standard curve of absorbance vs. cytokine concentration was created from the sample standards. Relative cytokine presence was calculated for each orgnaoid-monolayer sample using the standard curve.

### Statistical Analysis.

Data was analyzed through SPSS (IBM, Armonk, NY). Mean particle size was evaluated as mean+/−1 standard deviation and relative M cell induction measured via cell counting and was represented as sample means+/−2 standard error of the means (SEM) (n=3 per treatment group). Independent t-tests (two-tailed) were utilized to evaluate M cell induction, where p values <0.05 were considered significant (*p<0.05, ** p<0.005, *** p<0.001). TEER values were represented as sample means+/−SEM (n=48). Cytokine levels were extrapolated through SPSS. A two-way ANOVA test was used for comparison: p<0.05 were considered significant (*p<0.05, ** p<0.01, *** p<0.001).

### Characterization of Particles

Plastic particles used in the systems and methods may be diluted in DMEM and detected using fluorescence microscopy and SEM to validate the manufacturing sizes and the fluorescence. Microscopy images taken at various levels of resolution exhibit significant fluorescence signals from different sizes of particles confirming the fluorescence labels on the particles. In addition, a uniform dispersion of particles without significant aggregation was detected in DMEM (validated by fluorescence imaging and images were omitted can be provided to a patent office, if needed). SEM images further confirmed that all particles had a spherical shape (images omitted but can be provided to a patent office, if needed). To statistically evaluate particle size, ImageJ and SPSS (e.g., imaging and statistical software) were used to analyze the means, standard deviations, and ranges of particle sizes in the SEM images (Table 1). Mean particle sizes were equivalent to labeled specificities (Table 1, Column 3). The particle sizes ranged from 28.56 to 31.82 nm for 30 nm NPs, from 97.13 to 104.25 nm for 100 nm, from 494.92 to 503.89 nm for 500 nm, and from 0.870 to 1.25 μm for the 1 μm MPs, respectively (Table 1, Column 5).

TABLE 1
Characterization and validation of the parameters
of polystyrene MPs and NPs by SEM image analysis

| PS MPs | | Mean particle | | Standard | | Particle | |
|---|---|---|---|---|---|---|---|
| and NPs | N | size | | deviation | | size range | |
| 30 nm | 25 | 30.06 | nm | ±0.63 | nm | 28.56-31.82 | nm |
| 100 nm | 50 | 99.72 | nm | ±1.61 | nm | 97.13-104.25 | nm |
| 500 nm | 25 | 500.06 | nm | ±2.65 | nm | 494.92-503.89 | nm |
| 1 μm | 100 | 1.02 | μm | ±0.08 | μm | 0.87-1.25 | μm |

| PS MPs | | Mean particle | | Standard | | Particle | |
|---|---|---|---|---|---|---|---|
| and NPs | N | size | | deviation | | size range | |
| 30 nm | 25 | 30.06 | nm | ±0.63 | nm | 28.56-31.82 | nm |
| 100 nm | 50 | 99.72 | nm | ±1.61 | nm | 97.13-104.25 | nm |
| 500 nm | 25 | 500.06 | nm | ±2.65 | nm | 494.92-503.89 | nm |
| 1 μm | 100 | 1.02 | μm | ±0.08 | μm | 0.87-1.25 | μm |

Establishment and Characterization of Human Intestinal Organoid Derived Monolayers with and without Induced M Cells

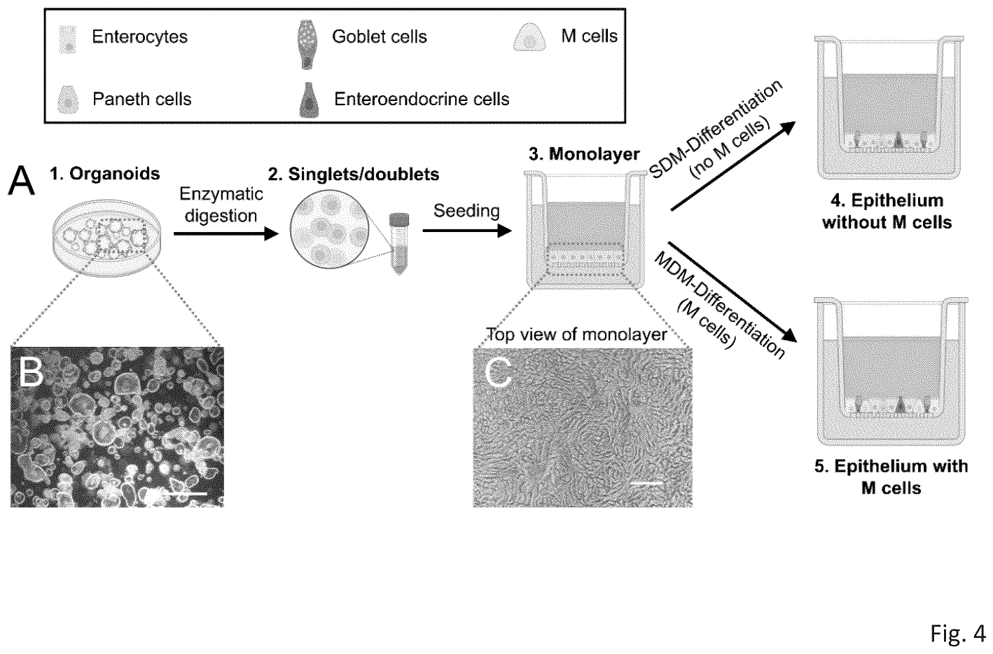

Human intestinal organoids (FIG. 4A-B) derived from ileum biopsy specimens were subcultured for monolayer generation. For monolayer preparation, organoids were enzymatically dissociated into single cells and seeded onto transwell inserts. Monolayers reached confluence 1-2 days post seeding (FIG. 4C). After that, the monolayers were either treated with Standard Differentiation Medium (SDM) or M-cell Differentiation Medium (MDM) to establish intestinal epithelia both with and without M cells (FIG. 4A). SDM and MDM have been widely utilized to generate well-differentiated epithelia derived from intestinal organoids. Organoid-derived epithelial monolayers treated with SDM for 3 days simultaneously differentiated into enterocytes, goblet cells, Paneth cells, enteroendocrine cells, while monolayers supplemented with MDM basolaterally for 6 days differentiated into enterocytes, goblet cells, Paneth cells, enteroendocrine cells, and M cells.

To confirm that these different major cell types were present in the differentiated monolayers, we collected and stained the SDM-differentiated monolayers at day 3 and day 6 post-differentiation to confirm the presence of relevant marker proteins. As expected, SDM-differentiated cell cultures expressed sucrase-isomaltase signal in the apical region of enterocytes throughout the monolayers, the mucin 2 (MUC2) staining in a scattering of goblet cells, the Lysozyme expression by Paneth cells, and the induction chromogranin A (ChgA) positive EECs. Furthermore, immunostaining also revealed the typical “chicken wire” staining pattern of ZO-1 tight junction protein, suggesting the individual mature epithelial cells are joined to their neighbors to assemble the tight junction network in the epithelium for maintenance of epithelial barrier integrity. A similar staining pattern of the above-mentioned markers was observed in MDM-differentiated epithelial monolayers (data not shown). To determine if MDM successfully induced M cells along with the major epithelial cell types in the monolayer cultures, immunofluorescence using M cell specific cell surface marker, glycoprotein 2 (GP2), was performed on MDM-differentiated epithelial monolayers. Immunofluorescence analysis showed that a significant amount of the M cells developed in the monolayers 6 days after treatment with MDM, as shown by the immunostaining for GP2. Moreover, by SEM, the differentiated monolayers displayed an apical-basolateral polarization of epithelial cells indicated by the presence of tightly packed microvilli at the apical side with random distribution of “bald” M cells with disorganized stubby microvilli along their apical border. Overall, these observations indicated that organoid-derived epithelial monolayers properly differentiated into the major intestinal epithelial cell types without or with M cells using SDM or MDM.

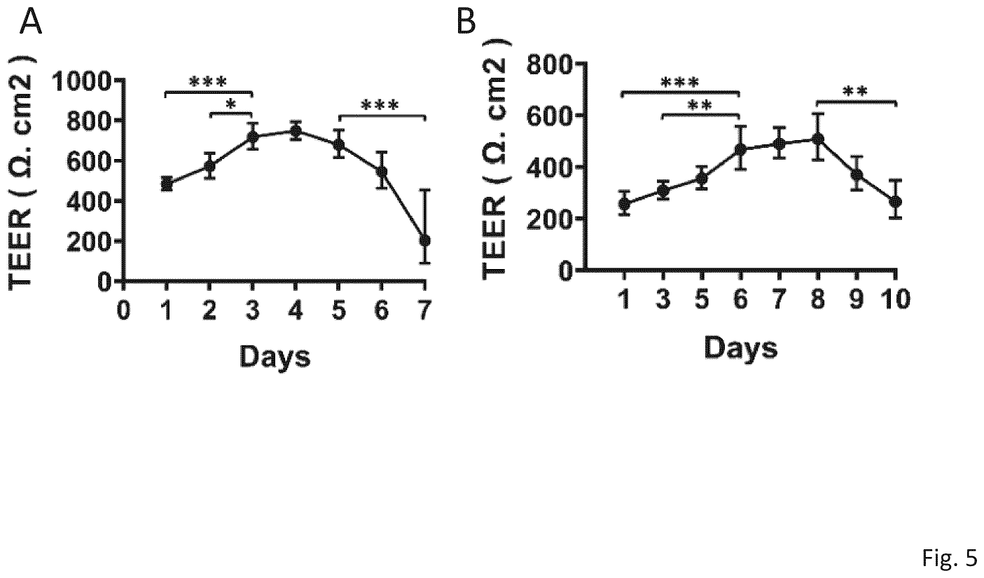

Differentiation of the organoid-derived monolayers was further evidenced by the significantly increased TEER values after switching to differentiation media in SDM- and MDM-differentiated epithelial monolayers (FIGS. 5A-B). Fully differentiated SDM-treated cultures possessed a TEER between 627 and 726 Ω*cm² (FIG. 5A); while fully differentiated MDM-treated cultures featured a TEER between 396-495 Ω*cm² (FIG. 5B). TEER values are strong indicators of epithelial maturation and integrity of the cellular barrier.

### Cellular Binding, Uptake and Translocation of PS MPs and NPs in Human Intestinal Epithelia

Next, we investigated the potential cytotoxic effects of PS MPs and NPs on human intestinal epithelial cells. Towards this goal, we first explored the interactions between the plastic particles and the gastrointestinal epithelium by studying the cellular binding, uptake and translocation of the particles by the intestinal epithelium. The two different intestinal organoid-derived epithelial models, as established previously herein, were employed: the SDM-differentiated monolayers (enterocytes, goblet cells, Paneth cells, EECs) and MDM-differentiated monolayers (enterocytes, goblet cells, Paneth cells, EECs, and M cells). After the cell seeding and differentiation with SDM or MDM (FIG. 6), different sizes of spherical fluorescent PS particles (1 μm, 500 nm, 100 nm, 30 nm) were applied on the apical side of the intestinal epithelia at a concentration of 500 μg/mL to explore particle uptake and transport. We started dosing the monolayers at 3 days post differentiation in SDM and 6 days post differentiation in MDM, as TEER values peaked at about 3 and 6 days after the differentiation was induced in SDM and MDM systems, respectively, and stabilized after that (FIGS. 5A-B). TEER value is a strong indicator of epithelial cell barrier integrity before evaluation for transport of particles. Samples from two different culture models were harvested 24, 48, 72, 96 hours post dosing (equals to days 4, 5, 6, 7 in the SDM culture, and days 7, 8, 9, 10 in the MDM culture, respectively) and prepared for confocal microcopy and SEM to detect the binding and trace the location of the MPs and NPs in the monolayer cultures. At 24 hours, all MPs and NPs bound to the intestinal epithelial cell surface and M cells (images not included, but can be provided to a patent office, if needed), which is a prerequisite for cellular uptake. Exposure to the plastic particles did not significantly affect tight junction formation demonstrated by ZO-1 expression. After 48 hours, in SDM-differentiated monolayers, particle sizes≤500 nm were imaged “nestling” or being enveloped by surrounding microvilli which may suggest these particles tend to cross the apical microvilli brush border of the epithelial cells (images excluded, but can be provided to a patent office, if needed), whereas in the MDM-differentiated monolayers, it was found that larger sized particles (1 μm, 500 nm) tended to be more specifically aggregated on and bound to the surface of M cells (images excluded, but can be provided to a patent office, if needed).

The intracellular uptake and translocation of MPs and NPs in the culture systems were tracked for up to 96 hours by confocal laser scanning microscopy (images omitted, but can be provided to a patent office, if needed). The three-dimensional image reconstruction of x-z cross-section from confocal sequential acquisition of the fluorescence signals from different planes enabled particle tracing at varying cellular depths. In the SDM-differentiated monolayers, over the course of 96 hours, the 1 μm MP group were mainly observed on the apical side of the intestinal epithelium, suggesting they did not cross the epithelia barriers. For the 500 nm MP group, at 96 hours post dosing, most of the particle fluorescence signals were still detected on the apical surface of the intestinal epithelium with occasional translocation across the epithelium, indicating no obvious particle translocation. For the 100 nm NP group, at 96 hours, particle signals started to appear inside some of the epithelial cells and on the basal side of the epithelium, suggesting cellular uptake and penetration of NPs. In the 30 nm NP group, at 96 hours, large numbers of particles were found on the basal side of the epithelium, suggesting that after cellular binding, the 30 nm NPs were taken up and translocated from the apical to the basal side of the epithelium. The particle tracking results demonstrated an MP/NP size-dependent uptake and translocation, with the highest total for PS uptake for the smallest NPs (30 nm). In the MDM-differentiated monolayers containing M cells, similar intracellular uptake and translocation patterns of 100 nm and 30 nm NPs were identified in the epithelium through 48 hours (data not shown). However, at 72 hours, obvious translocation of 100 nm and 30 nm NPs to the basal side of the epithelia was observed, followed by the degradation of epithelial tight junctions (ZO-1) (100 nm) and the lifting of monolayers from the transwell inserts (30 nm) at 96 hours post particle exposure. Furthermore, 1 μm and 500 nm particles preferentially internalized and accumulated in M cells (images omitted, but can be provided to a patent office, if needed).

### Cytotoxicity Effect of PS MPs and NPs in Human Intestinal Epithelia

Knowing that different sizes PS MPs and NPs can interact with and cross the human intestinal epithelia, we next assessed their cytotoxic effects by using different concentrations and exposure periods in the human intestinal epithelia. In this study, the toxicity assessment was conducted using two different assays: TEER—to examine epithelial integrity and ELISA—to measure cytokine secretion (inflammatory responses). For cytokine profiling, the release of proinflammatory cytokines interleukin-6 (IL-6), IL-8, tumor necrosis factor-alpha (TNF-α) and an anti-inflammatory cytokine TGF-β1 were analyzed. The two different intestinal organoid-derived epithelial models (with and without induced M cells) were compared. The cytotoxic response of cells treated with MPs and NPs (1 μm, 500 nm, 100 nm, and 30 nm) for 24, 48, 72 and 96 hours was investigated using particle concentrations ranging from 250-1000 μg/mL (250, 500, 1000 μg/mL).

Barrier Integrity and Cytokine Secretion in Particle-Exposed Epithelia without Induced M Cells

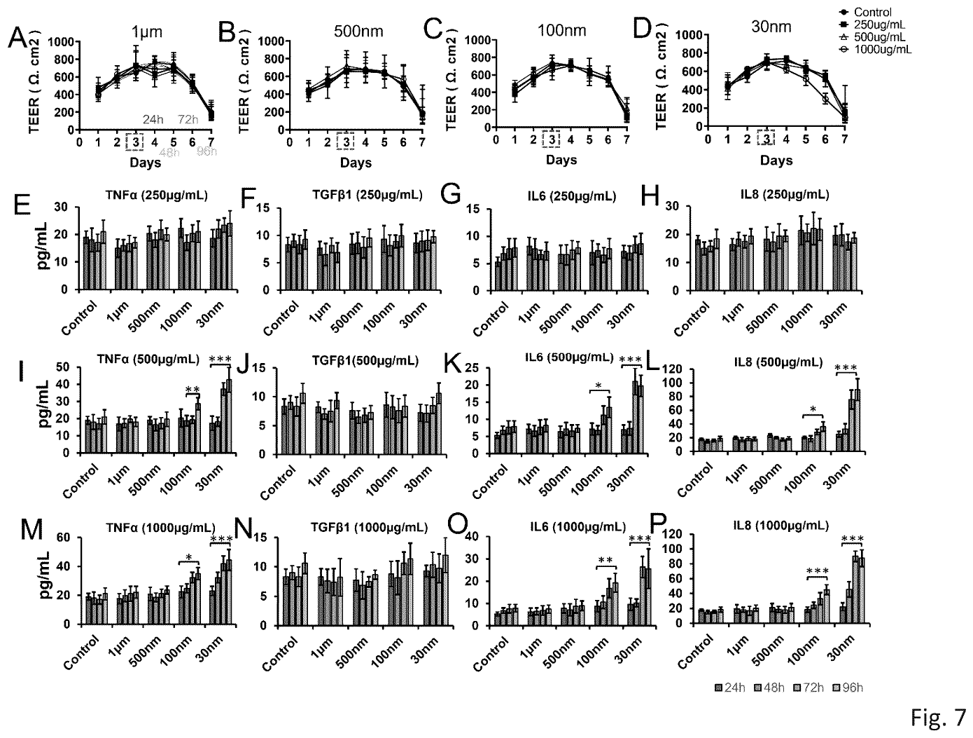

The TEER values of SDM-differentiated monolayers treated with 250, 500, 1000 μg/mL of 1 μm, 500 nm, 100 nm particles remained relatively constant and equivalent to the control over the duration of the exposure (FIGS. 7A-C), suggesting the particles did not substantially and adversely affect the intestinal barrier integrity. For the 30 nm group (FIG. 7D), cellular integrity was not altered by 250 and 500 μg/mL particles until 48 hours (day 5 in culture) post dosing. At 72 hours, 30 nm NPs induced a slight decline in TEER values at 1000 μg/mL.

The levels of cytokines (TNF-α, TGF-β1, IL-6, IL-8) in all exposure groups of MPs and NPs (FIGS. 7E-H) were determined and no significant increase in cytokine secretions were induced from any of the particle groups at 250 μg/mL compared to the control groups. However, at 500 and 1000 μg/mL, secretion of TNFα, IL-6 and IL-8 was significantly increased in the 100 nm and 30 nm NPs after 96 or 72 hours of exposure (** p<0.01, FIGS. 7I, K and L). By extrapolation, these results demonstrated that the smaller sizes (100 nm and 30 nm) of the PS NPs at concentrations greater than 500 μg/mL might induce the release of pro-inflammatory cytokines (TNF-α, IL-6 and IL-8) in the human intestinal epithelial cells.

Barrier Integrity and Cytokine Secretion in Particle-Exposed Epithelia with M Cells

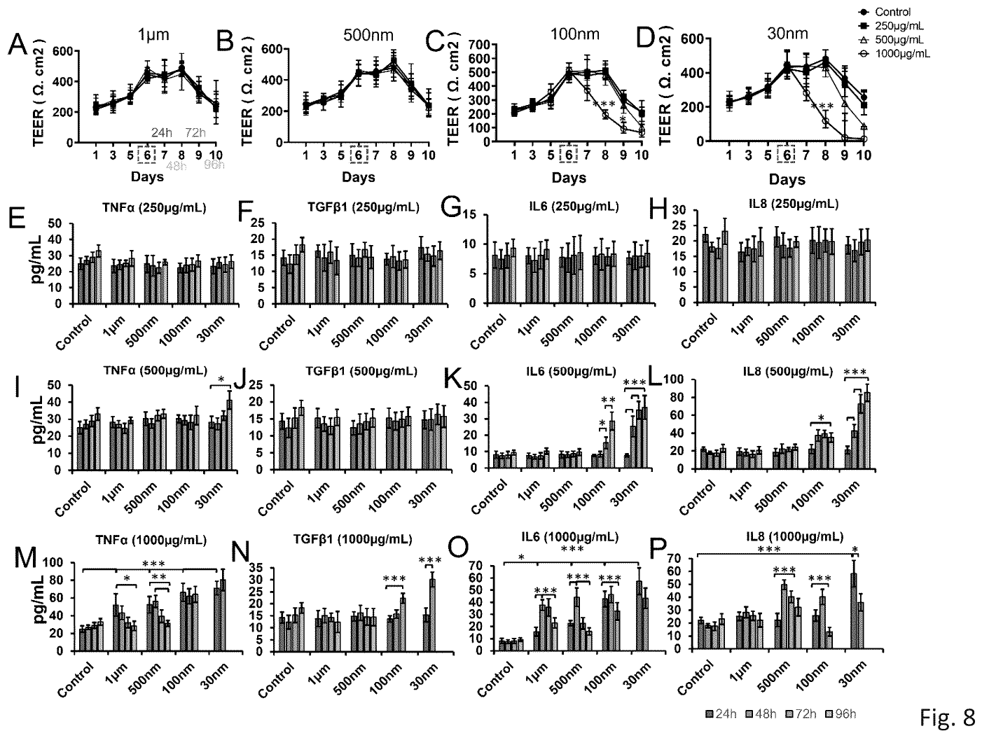

According to the TEER analysis, the epithelial integrity of MDM-differentiated monolayers was not significantly impacted by 1 μm and 500 nm particles at any concentration (FIGS. 10A-B) but was impacted by 100 nm and 30 nm PS particles at 250 and 500 μg/mL over time (FIGS. 8C-D). However, compared to the control group, a significant decrease (*p<0.05) in TEER value was observed in monolayers that were exposed to 100 nm and 30 nm NP groups at 1000 μg/mL for 48 hours (FIGS. 8C-D), indicating impaired epithelial barrier function was caused by the exposure to 1000 μg/mL 100 nm and 30 nm particles.

For cytokine production, compared to the control groups, no statistically significant changes were detected for all particle sizes in 250 μg/mL group throughout the exposure course. Increased release of cytokines (TNF-α, IL-6, IL-8) in the 500 μg/mL group were detected and sustained in the 100 nm and 3 0 nm NPs across various time points (FIGS. 8I, K, and L) with no obvious changes in TGFβ1 (FIG. 8J). Specifically, 500 μg/mL 100 nm NPs triggered a higher release of IL-6 at 72 hours post exposure (FIG. 8K) and IL-8 at 48 hours (FIG. 8L). Comparatively, the 30 nm NPs significantly triggered TNF-α secretion at 96 hours post exposure and IL-6 and IL-8 secretions as early as 48 hours (FIGS. 8K-L). In the 1000 μg/mL groups, acute releases of TNF-α and IL-6 by cells were found in all different sized MPs and NPs evidenced by the rapid and excessive production at 24 hours post dosing (FIGS. 8M and 10O). The acute release of IL-8 at 24 hours was observed in the 30 nm group (FIG. 8P). Detectable levels of IL-8 production in 500 nm and 100 nm groups were not observed until 48 hours post exposure (FIGS. 8O and 8P). In these groups, the mean cytokine levels sustained or kept increasing from 24 to 48 hours but tended to decline after that. Notably, in 1000 μg/mL groups, the TGF-β1 concentrations were significantly elevated by 100 nm particles at 72 hours and by 30 nm particles at 48 hours (*p<0.05, FIG. 8N). Monolayers detached from the Transwell inserts in 100 nm groups at 96 hours and 30 nm groups at 72 hours and cytokine data from these groups were not collected.

### Discussion

After targeting the cells, different sized particles internalize and pass through the intestinal epithelium by different pathways and mechanisms for them to undergo intracellular transport via regular epithelial cells and M cells, and then exit the cells at the basolateral membrane to complete the absorption process. Accordingly, we generated two physiologically relevant epithelial organoid monolayer models following well-established differentiation protocols (FIG. 5): SDM-induced monolayer without M cells (enterocytes, goblet cells, EECs, and Paneth cells) and MDM-induced monolayers with M cells (enterocytes, goblet cells, EECs, and Paneth cells, M cells). Our intention was to simulate in vivo cellular complexity for particle uptake and translocation to identify how different cell models respond to the exposure of MPs and NPs and ultimately decipher the contribution of M cells during these responses. Using immunostaining with various cell specific markers and SEM, we validated that the different protocols successfully induced the undifferentiated monolayers into mature intestinal epithelia with desired cell populations consisting of the major epithelial cell types without/with M cells.

Several studies using in vitro cell models have indicated that the size of particles plays a critical role in determining the efficiency of cellular uptake as well as the uptake pathway in different living human cells. For example, a size-dependent uptake has been observed for PS particles in human lung cells, astrocytoma cells, umbilical vein endothelial cells, intestinal cells (Caco-2). In all cases, smaller particles had a higher chance of penetrating the cell membrane, demonstrating larger adverse effects, and the particles had maximal cellular uptake in the size range of 30-50 nm. Our results showed that, in SDM-induced intestinal epithelia, all MPs and NPs homogeneously attached to and potentially entered via the apical side of the epithelial surface with a significant uptake only occurring at the particle size of 30 nm at 96 hours after exposure. In contrast, in the monolayers containing M cells, while the cellular internalization of the MPs and NPs was also highly dependent on the size, plastic particles larger than 500 nm preferentially aggregated in M cells and penetrated the monolayers through M cells. In addition, the incorporation of M cells generally increased the degree of translocation of MPs and NPs at all sizes. Particularly, 30 nm particles were randomly taken up by regular epithelial cells (enterocytes and secretory cells), whereas larger particles (500 nm and 1 μm) crossed the epithelium via M cells. This finding is supported by earlier in vivo data demonstrating that larger PS particles are taken up exclusively by M cells associated with Peyer's patch in rats and mice and in vitro data showing M-like cells from cancerous cell lines and M-cells induced from mouse intestinal organoids were strongly associated with higher uptake of PS NPs. A key characteristic of functional M-cells is that they can efficiently capture and transport large particles and bacteria across the epithelial barrier. In this regard, M cells induced from intestinal organoids exhibit similar functional characteristics to M-cells in vivo, confirming the potential of intestinal organoids as a platform for enriching and studying rare intestinal cell types, such as M cells, EECs, Tuft cells. Moreover, our results also agreed with the accepted mechanism of microparticle (i.e. >100 nm) uptake through transcytosis across M-cells located in the Peyer's patches and nanoparticle (i.e. <100 nm) uptake through other epithelial cells, such as enterocytes and goblet cells, by endocytosis or paracellular transport through tight junctions (only for particles smaller than 5 nm). These results suggest that the cellular translocation mechanism and efficiency of the PS MPs and NPs in human intestine significantly relies on the particle size.

After observing the differences in internalization behaviors with MPs and NPs in different sizes, we further evaluated two other experimental variables, concentration and exposure period, on the cytotoxic impact of the particles on intestinal epithelial cells by examining the epithelial barrier integrity using TEER and immune response using ELISA-based cytokine measurement (FIGS. 7 and 8). Given that MPs and NPs accumulate in the gut of several living organisms and that large plastic debris may even block the gastrointestinal tract in some severe cases, we decided on screening extreme effects of PS particles using high concentrations ranging from 250 to 1000 μg/mL. Many of the available studies have used similar concentration ranges to assess the biological effects of PS MPs and NPs in intestinal or other cell types, with some studies testing even higher particle doses (up to 2000 μg/mL).

During the plastic exposure experiments, we did not observe obvious changes in the TEER values in the SDM-induced epithelia exposed to 1 μm, 500 nm, 100 nm particles, independent of the concentrations (250 μg/mL, 500 μg/mL, and 1000 μg/mL), and exposure period (24, 48, 72 and 96 hours). Similar results were obtained in the MDM-induced epithelia treated with 1 μm and 500 nm MPs. This is in line with work from other groups employing either differentiated or undifferentiated Caco-2 cells, in which no or slight impact on cell viability and membrane integrity was found when exposed to PS particles (100 nm-5 μm). It is worth noting that the highest concentration tested in these studies was 200 μg/mL and the longest exposure time in the experiments was 48 hours.[1] As an exception, 5 μm PS-MPs inhibited the proliferation of Caco-2 cells in vitro in a time- and concentration-dependent pattern.[2] However, in that study Caco-2 cells were cultured in planar plastic substrate, which could affect the cell growth and differentiation and therefore lead to discrepancies in the results. Our data obtained from human derived intestinal organoids (with and without M cells) supplement the existing knowledge regarding PS particle toxicity by showing even higher concentrations of particles larger than 500 nm (500 nm and 1 μm) with prolonged exposure time (up to 96 hours) did not lead to obvious damage to the intestinal epithelial integrity. Additionally, in the exposure study, as the concentration of particles was increased to 1000 μg/mL, 30 nm NP exposure groups mildly induced decreases in TEER in SDM-induced epithelia, whereas both the 100 nm and 30 nm groups significantly decreased TEER in the MDM-induced monolayer starting from 48 hours of exposure. It is worth mentioning that, compared to the control groups, in the monolayers without M cells, cell viability did not seem to be significantly affected by MPs and NPs. However, intestinal monolayers containing M cells detached in the groups of 100 nm/96 hour, 30 nm/72 hour and 30 nm/96 hour at a concentration of 1000 μg/mL, indicating epithelial cell damage during exposure to smaller sized groups. This observation agrees with previous reports documenting that smaller sized nanoparticles are correlated with higher cell damage in both in vitro and in vivo models and that additionally, PS particles do not cause notable cytotoxicity in human skin dermal fibroblasts and blood cells at concentrations up to 500 μg/mL. Interestingly, both 30 nm and 100 nm groups resulted in more progressive reduction of TEER in the M cell containing epithelia.

The mechanisms of MPs and NPs cytotoxicity also involve stimulating the secretion of inflammatory cytokines. Cytokines represent a vast family of immunomodulatory substances typically including Interleukins (IL), Interferon (IFN), Growth Factor (GF), Tumor Necrosis Factor (TNF), and Chemokines (CKs). Maintaining a low-grade “physiological inflammation” is essential for normal tissue function. During infection or injury, multiple intestinal immune and non-immune cells release abundant counter-regulatory cytokines to enable gut immune homeostasis and tolerance. Dysfunction of cytokine release by the immune system in host epithelial cells has been implicated in inflammatory bowel disease (IBD), which affects approximately 3.1 million adults (1.3%) in the United States and more than 10 million people worldwide. Among different cytokines, the most studied members involved in intestinal immune inflammation comprise TNF-α, TGF-β1, IL-6 and IL-8. The upregulation of these cytokines are detected in the intestine tissue samples from patients with active IBD.

Because of the lack of standardized research models and high diversity of the MP and NPs (different size, shape, surface charging and polymer type) used in different studies, there are contradictory results derived from both in vivo and in vitro studies on whether MPs and MPs activate inflammatory reactions in the intestine.[3] For example, in mouse models, one study showed that after 21-weeks of exposure to environmentally relevant doses of large PS MPs (40-100 μm), mice displayed a clear intestinal inflammatory response with infiltration of immune cells. Similarly, intestinal barrier dysfunction and cell death was found in mice that had been administered PS particles in varying sizes (50 nm, 500 nm and 5000 nm) under single or co-exposure conditions in a size-dependent manner.[4] In contrast, 5 μm PS MPs alone caused minimal effects on the intestinal barrier of mice, but exposure to PS MPs resulted in histological injury, intestinal microbiota disorders, inflammation, and oxidative stress in mice with acute and chronic colitis.[5] A more recent study compared the effects of PS MPs (5 μm) on healthy mice and mice with intestinal immune imbalances. The authors concluded that PS MPs exposure only significantly stimulated the expression of inflammation factors (TNFα, IL-1B and IFN-γ) in mice with intestinal immune imbalance.[6] Data from in vitro human intestinal cell models also contributed to the conflicting results. Some studies have cited that 5 μm PS particles showed little influence on IL-8 and MCP-1 secretion in Caco-2 cells, however, IL-8 levels were enhanced by exposure to 100 nm particles at a concentration of 20 μg/mL.[7] Other studies highlighted that 3 and 10 μm gave rise to a large amount of reactive oxygen species (ROS) production, key signaling molecules in inflammatory disorders, in human intestinal cells in the short term.[8] In the current study, from the cytokine profiles, compared to the non-dosing control groups, virtually no cytokine release was induced until concentrations reached 500 μg/mL in both SDM- and MDM induced intestinal epithelia. Additionally, based on the general increased level of the cytokines in 500 μg/mL and 1000 μg/mL groups, the exposure of PS MPs and NPs induced stronger inflammatory reaction on monolayers with M cells than without M cells. For example, we found that under 1000 μg/mL, both 500 nm and 1 μm MPs significantly induced the secretion of TNF-α, IL-6 and IL-8 compared to the control group, which did not occur in the monolayers without M cells. The key role of the M cells is to sense, capture and deliver antigens/pathogens across the epithelium, thus triggering mucosal immune response. In our case, the larger particles (500 nm and 1 μm) were sensed, sampled, and transported through M cells (confirmed in our tracking assay) which led to production of pro-inflammatory cytokines for protection against potential antigens and pathogens. Furthermore, TGF-β1, not promoted in monolayers without M cells in exposure group, was significantly upregulated in 100 nm/72 hour and 30 nm/48 hour exposure groups. As a pleiotropic cytokine, the role of TGF-β1 in IBD is still under debate. However, most researchers believe that TGF-β inhibits the intestinal inflammatory responses to luminal bacteria and food antigens, thus achieving immune tolerance. In this scenario, when exposed to small PS particles, M cells contribute to the activation of the specific immunological pathways (TGF-β signaling pathway) to downregulate excessive inflammatory cytokine release and ameliorate epithelial inflammation caused by particle exposure. However, we also found epithelial cell damage in the groups of 100 nm/96 hour, 30 nm/72 hour and 30 nm/96 hour at a concentration of 1000 μg/mL in the monolayers containing M cells, which did not occur in the monolayer without M cells. This may indicate that the anti-inflammatory activity against small particles triggered by M cells and other epithelial cells was not intense enough to suppress the “cytokine storm” resulting from the sudden and acute increase in levels of TNF-α, IL-6 and IL-8 in the groups with cell damage (FIGS. 8M, 8O, and 8P). Other immune sensors, such as B cells in gut-associated lymphoid tissue (GALT), macrophages, dendritic cells, and mast cells should be involved to reach native immune protection. Based on these findings, we concluded that the ingestion of PS MP and NP suggests the potential towards IBD-related risks via the induced inflammatory cytokines associated with IBD. Furthermore, M cells induced from human intestinal organoids were able to imitate the biological functions of native M cells and that the MDM-induced monolayer represents an improved intestinal follicle-associated epithelium to study MPs and NPs transport by M cells.

### REFERENCES

1. J. Domenech, A. Hernandez, L. Rubio, R. Marcos and C. Cortes, Interactions of polystyrene nanoplastics with in vitro models of the human intestinal barrier. Arch Toxicol. 2020; 94:2997-3012
2. S. Wu, M. Wu, D. Tian, L. Qiu and T. Li, Effects of polystyrene microbeads on cytotoxicity and transcriptomic profiles in human Caco-2 cells. Environ Toxicol. 2020; 35:495-506
3. Y. Jin, L. Lu, W. Tu, T. Luo and Z. Fu, Impacts of polystyrene microplastic on the gut barrier, microbiota and metabolism of mice. Sci Total Environ. 2019; 649:308-317
4. B. Liang, Y. Zhong, Y. Huang, X. Lin, J. Liu, L. Lin, et al., Underestimated health risks: polystyrene micro- and nanoplastics jointly induce intestinal barrier dysfunction by ROS-mediated epithelial cell apoptosis. Particle and Fibre Toxicology. 2021; 18:20
5. T. Luo, D. Wang, Y. Zhao, X. Li, G. Yang and Y. Jin, Polystyrene microplastics exacerbate experimental colitis in mice tightly associated with the occurrence of hepatic inflammation. Sci Total Environ. 2022; 844:156884
6. S. Liu, H. Li, J. Wang, B. Wu and X. Guo, Polystyrene microplastics aggravate inflammatory damage in mice with intestinal immune imbalance. Sci Total Environ. 2022; 833:155198
7. S. Liu, X. Wu, W. Gu, J. Yu and B. Wu, Influence of the digestive process on intestinal toxicity of polystyrene microplastics as determined by in vitro Caco-2 models. Chemosphere. 2020; 256:127204
8. G. Visalli, A. Facciola, M. Pruiti Ciarello, G. De Marco, M. Maisano and A. Di Pietro, Acute and Sub-Chronic Effects of Microplastics (3 and 10 microm) on the Human Intestinal Cells HT-29. Int J Environ Res Public Health. 2021; 18.
9. Chen, Y.; Zhou, W.; Roh, T.; Estes, M. K.; Kaplan, D. L., In vitro enteroid-derived three-dimensional tissue model of human small intestinal epithelium with innate immune responses. PLOS One 2017, 12 (11), e0187880.
10. Lin, S. C.; Qu, L.; Ettayebi, K.; Crawford, S. E.; Blutt, S. E.; Robertson, M. J.; Zeng, X. L.; Tenge, V. R.; Ayyar, B. V.; Karandikar, U. C.; Yu, X.; Coarfa, C.; Atmar, R. L.; Ramani, S.; Estes, M. K., Human norovirus exhibits strain-specific sensitivity to host interferon pathways in human intestinal enteroids. Proc Natl Acad Sci USA 2020, 117 (38), 23782-23793.
11. Murakami, K.; Tenge, V. R.; Karandikar, U. C.; Lin, S. C.; Ramani, S.; Ettayebi, K.; Crawford, S. E.; Zeng, X. L.; Neill, F. H.; Ayyar, B. V.; Katayama, K.; Graham, D. Y.; Bieberich, E.; Atmar, R. L.; Estes, M. K., Bile acids and ceramide overcome the entry restriction for GII.3 human norovirus replication in human intestinal enteroids. Proc Natl Acad Sci USA 2020, 117 (3), 1700-1710.
12. Saxena, K.; Simon, L. M.; Zeng, X. L.; Blutt, S. E.; Crawford, S. E.; Sastri, N. P.; Karandikar, U. C.; Ajami, N. J.; Zachos, N. C.; Kovbasnjuk, O.; Donowitz, M.; Conner, M. E.; Shaw, C. A.; Estes, M. K., A paradox of transcriptional and functional innate interferon responses of human intestinal enteroids to enteric virus infection. Proc Natl Acad Sci USA 2017, 114 (4), E570-E579.
13. Ettayebi, K.; Crawford, S. E.; Murakami, K.; Broughman, J. R.; Karandikar, U.; Tenge, V. R.; Neill, F. H.; Blutt, S. E.; Zeng, X. L.; Qu, L.; Kou, B.; Opekun, A. R.; Burrin, D.; Graham, D. Y.; Ramani, S.; Atmar, R. L.; Estes, M. K., Replication of human noroviruses in stem cell-derived human enteroids. Science 2016, 353 (6306), 1387-1393.
14. Fasciano, A. C.; Blutt, S. E.; Estes, M. K.; Mecsas, J., Induced Differentiation of M Cell-like Cells in Human Stem Cell-derived Ileal Enteroid Monolayers. J Vis Exp 2019, (149): 10.3791/59894.
15. Noel, G.; Baetz, N. W.; Staab, J. F.; Donowitz, M.; Kovbasnjuk, O.; Pasetti, M. F.; Zachos, N. C., A primary human macrophage-enteroid co-culture model to investigate mucosal gut physiology and host-pathogen interactions. Sci Rep 2017, 7, 45270.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

1. A system for providing ex vivo approximation of micro- and/or nanoparticle oral toxicity in humans, the system comprising:
an ex vivo human intestinal model organoid comprising an epithelial layer that separates an apical volume and a basal volume of the ex vivo human intestinal model organoid;
a bioreactor for maintaining the ex vivo human intestinal model organoid under physiologically relevant culture conditions;
a particle administration inlet adapted to receive a predefined quantity of micro- and/or nanoparticles and to introduce the predefined quantity to the apical volume of the ex vivo human intestinal model organoid according to a predefined dosing regimen; and
an analytical platform adapted to perform at least one of the following: interrogate the ex vivo human intestinal model organoid or associated culture media within the bioreactor; or to receive and interrogate the at least one of ex vivo human intestinal model organoid or the associated culture media after removal from the bioreactor; and
a processor and a memory, the memory having stored thereon instructions that, when executed by the processor, cause the processor to:
a) optionally send a signal to the particle administration inlet, thereby automatically controlling the predefined dosing regimen;
b) at one or more predetermined times relative to the predefined dosing regimen, using the analytical platform, at least one of imaging the ex vivo human intestinal model organoid to identify localization of the predefined quantity of at least one of microparticles- or nanoparticles, measuring tight-junction degradation within the ex vivo human intestinal model organoid optionally by measuring ZO-1 expression levels, measuring epithelial integrity of the ex vivo human intestinal model organoid, or measuring cytokine upregulation within the ex vivo human intestinal model organoid; and
c) generating a report including acquired data relating to the localization of the predefined quantity of at least one of microparticles- or nanoparticles within at least one of the ex vivo human intestinal model organoid, the tight-junction degradation within the ex vivo human intestinal model organoid, the epithelial integrity of the ex vivo human intestinal model organoid, or the cytokine upregulation within the ex vivo human intestinal model organoid, wherein the ex vivo human intestinal model organoid comprises either: an M cell concentration of between 0.1% and 10% within the epithelial layer; or an initial transepithelial electrical resistance of between 0 Ω*cm² and 1500 Ω*cm².

2. The system of claim 1, wherein the particle administration inlet is an automated particle hopper.

3. The system of claim 1, wherein the analytical platform comprises at least one of a transepithelial electrical resistometer, a confocal laser scanning microscope, a scanning electron microscope, an assay, an ELISA assay for cytokine upregulation, an ELISA assay for tumor necrosis factor (TNF)-α, an ELISA assay for tumor growth factor (TGF)-β1, an ELISA assay for interleukin (IL)-6, or an ELISA assay for IL-8.

4.-12. (canceled)

13. The system of claim 1, wherein the bioreactor is coupled to a culture media reservoir and the system is adapted to at least one of regenerate or circulate the associated culture media within the bioreactor to maintain the physiologically relevant culture conditions.

14. The system of claim 1, the system further comprising a bioreactor supernatant sampler operable to extract a culture media supernatant sample from the bioreactor.

15. The system of claim 14, wherein the bioreactor supernatant sampler is further operable to deliver the culture media supernatant sample to the analytical platform or the assay or the ELISA assay.

16. A method of ex vivo approximation of at least one of microparticle oral toxicity- or nanoparticle oral toxicity using an ex vivo human intestinal model organoid comprising an epithelial layer that separates an apical volume and a basal volume of the ex vivo human intestinal model organoid, the method comprising:
a) introducing a predefined quantity of at least one of microparticles- or nanoparticles into the apical volume of the ex vivo human intestinal model organoid in a predefined dosing regimen under physiologically relevant culture conditions;
b) at one or more predetermined times relative to the predefined dosing regimen, imaging the ex vivo human intestinal model organoid to identify localization of the predefined quantity of at least one of microparticles- or nanoparticles, measuring tight-junction degradation within the ex vivo human intestinal model organoid by at least one of measuring ZO-1 expression levels, measuring epithelial integrity of the ex vivo human intestinal model organoid, or measuring cytokine upregulation within the ex vivo human intestinal model organoid; and
c) generating a report including acquired data relating to the localization of the predefined quantity of at least one of microparticles- or nanoparticles within the ex vivo human intestinal model organoid, the tight-junction degradation within at least one of the ex vivo human intestinal model organoid, the epithelial integrity of the ex vivo human intestinal model organoid, or the cytokine upregulation within the ex vivo human intestinal model organoid, wherein the ex vivo human intestinal model organoid comprises either: an M cell concentration of between 0.1% and 10% within the epithelial layer; or an initial transepithelial electrical resistance of between 0 Ω*cm² to 1500 Ω*cm².

17. (canceled)

18. The system of claim 1, the instructions, when executed by the processor, further causing the processor to: associate at least one of the localization, the tight-junction degradation, the epithelial integrity, or the cytokine upregulation with a predicted in vivo toxicity for the microparticles- or nanoparticles.

19.-22. (canceled)

23. The system of claim 1, wherein the ex vivo human intestinal model organoid comprises the M cell concentration of between 2% and 8% within the epithelial layer.

24. The system of claim 1, wherein the ex vivo human intestinal model organoid comprises the M cell concentration of between 4% and 6% within the epithelial layer.

25. (canceled)

26. The system of claim 1 wherein the ex vivo human intestinal model organoid comprises the initial transepithelial electrical resistance of between 200 Ω*cm² to 1200 Ω*cm².

27. The system of claim 1, wherein the ex vivo human intestinal model organoid comprises the initial transepithelial electrical resistance of between 400 Ω*cm² to 600 Ω*cm².

28. The system of claim 1, wherein the ex vivo human intestinal model organoid is a two-dimensional human intestinal model organoid.

29. The system of claim 1, wherein the ex vivo human intestinal model organoid is a three-dimensional human intestinal model organoid.

30. The system of claim 1, wherein the ex vivo human intestinal model organoid is patient-specific and the report includes patient-specific data or patient-specific predictions.

31. (canceled)

32. The system of claim 1, wherein the imaging is performed using a scanning electron microscope, a confocal laser scanning microscope, or a combination thereof.

33.-37. (canceled)

38. The system of claim 1, wherein measuring the epithelial integrity includes measuring transepithelial electrical resistance.

39.-46. (canceled)

47. The system of claim 1, wherein the at least one of microparticles- or nanoparticles are at least partly polymeric.

48. The system of claim 1, wherein the at least one of microparticles or nanoparticles are at least partly metallic.

49. The system of claim 1, wherein the ex vivo human intestinal model organoid is composed of cells at least one of grown or differentiated on a silk fibroin-based tissue scaffold.
